(19) United States
(12) Patent Application Publication
(10) Pub. No.: US 2022/0243543 A1
(43) Pub. Date: Aug. 4, 2022

(54) METHOD AND SYSTEM FOR DRILLING FLUID CONDITION MONITORING

(71) Applicant: SAUDI ARABIAN OIL COMPANY, Dhahran (SA)

(72) Inventors: Mahmoud F. Abughaban, Dhahran (SA); Ashok Kumar Santra, The Woodlands, TX (US)

(73) Assignees: SAUDI ARABIAN OIL COMPANY, Dhahran (SA); ARAMCO SERVICES COMPANY, Houston, TX (US)

(21) Appl. No.: 17/167,501

(22) Filed: Feb. 4, 2021

(51) Int. Cl. E21B 21/01; B01F 15/00; B01F 3/12; B01F 3/08; G01N 33/28

(52) U.S. Cl. CPC E21B 21/01; B01F 15/00246; B01F 15/00233; B01F 15/0022; B01F 15/00344; B01F 2215/0081; B01F 3/0803; B01F 3/088; B01F 3/1271; G01N 33/2823; B01F 3/1207

(57) ABSTRACT

A method may include supplying water for a mud mixture to a mixing tank according to a predetermined volume. The method may further include supplying, using a rheological sensor, a viscosifier to the mud mixture in the mixing tank until the mud mixture achieves one or more predetermined rheological values. The method may further include supplying, using a density sensor, a weighting agent to the mud mixture in the mixing tank until the mud mixture achieves a predetermined specific gravity value. The method may further include supplying, using a pH sensor, a buffering agent to the mud mixture in the mixing tank until the mud mixture achieves a predetermined pH value to produce a drilling fluid.

### BACKGROUND

Drilling fluid, also called drilling mud, may be a heavy, viscous fluid mixture that is used in oil and gas drilling operations to carry rock cuttings from a wellbore back to the surface. Drilling mud may also be used to lubricate and cool a drill bit. The drilling fluid, by hydrostatic pressure, may also assist in preventing the collapse of unstable strata into the wellbore as well as the intrusion of water from stratigraphic formations proximate the wellbore.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method that includes obtaining, by a computer processor, a selection of various drilling fluid properties from a user device. The drilling fluid properties include a predetermined specific gravity value. The method further includes transmitting, by the computer processor, a first command to supply a mixing tank automatically with a viscosifier to produce a mud mixture. The method further includes transmitting, by the computer processor, a second command that causes a weighting agent to be supplied to the mud mixture in the mixing tank until a density sensor coupled to the mixing tank determines that the mud mixture corresponds to the predetermined specific gravity value. The method further includes transmitting, by the computer processor, a third command that causes the mud mixture to circulate within a wellbore.

In general, in one aspect, embodiments relate to a system that includes a drilling fluid processing system including a mixing tank, a feeder, a user device, and a first control system. The system further includes a drilling system coupled to the drilling fluid processing system and a wellbore, where the drilling system includes a second control system and a drill string. The second control system circulates drilling fluid within the wellbore. The first control system includes a computer processor. The first control system obtains a selection of various drilling fluid properties from the user device. The drilling fluid properties include a predetermined pH value and a predetermined specific gravity value. The first control system transmits a first command to supply a mixing tank automatically with a viscosifier to produce a mud mixture. The first control system transmits a second command that causes a weighting agent to be supplied to the mud mixture in the mixing tank to produce a drilling fluid. The weighting agent is supplied until a density sensor coupled to the mixing tank determines that the mud mixture corresponds to the predetermined specific gravity value. The first control system transmits, to the second control system, a third command that causes the drilling fluid to circulate within a wellbore.

In general, in one aspect, embodiments relate to a non-transitory computer readable medium storing instructions executable by a computer processor. The instructions obtain a selection of various drilling fluid properties from a user device. The drilling fluid properties include a predetermined specific gravity value. The instructions further transmit a first command to supply a mixing tank automatically with a viscosifier to produce a mud mixture. The instructions further transmit a second command that causes a weighting agent to be supplied to the mud mixture in the mixing tank until a density sensor coupled to the mixing tank determines that the mud mixture corresponds to the predetermined specific gravity value. The instructions further transmit a third command that causes the mud mixture to circulate within a wellbore.

In general, in one aspect, embodiments relate to a method that includes supplying water for a mud mixture to a mixing tank according to a predetermined volume. The method further includes supplying, using a rheological sensor, a viscosifier to the mud mixture in the mixing tank until the mud mixture achieves one or more predetermined rheological values. The method further includes supplying, using a density sensor, a weighting agent to the mud mixture in the mixing tank until the mud mixture achieves a predetermined specific gravity value. The method further includes supplying, using a pH sensor, a buffering agent to the mud mixture in the mixing tank until the mud mixture achieves a predetermined pH value to produce a drilling fluid.

In general, in one aspect, embodiments relate to a system that includes a mixing tank and various sensors coupled to the mixing tank, where the sensors include a rheological sensor, a density sensor, and a pH sensor. The system further includes a first control system coupled to the mixing tank and the sensors. The system further includes a drilling system coupled to the mixing tank and a wellbore, where the drilling system includes a second control system and a drill string. The second control system circulates drilling fluid within the wellbore. The first control system supplies water for a mud mixture to a mixing tank according to a predetermined volume. The first control system supplies, using the rheological sensor, a viscosifier to the mud mixture in the mixing tank until the mud mixture achieves a predetermined rheological value. The first control system supplies, using the density sensor, a weighting agent to the mud mixture in the mixing tank until the mud mixture achieves a predetermined specific gravity value. The first control system supplies, using the pH sensor, a buffering agent to the mud mixture in the mixing tank until the mud mixture achieves a predetermined pH value to produce a drilling fluid.

In general, in one aspect, embodiments relate to a method that includes supplying diesel or mineral oil to a mixing tank according to a predetermined volume to produce a mud mixture. The method further includes supplying an inorganic viscosifier, an emulsifier, a wetting agent, lime, a rheology modifier, a brine, and a fluid loss control additive to the mud mixture in the mixing tank. The method further includes supplying, using a rheological sensor, a polymeric viscosifier to the mud mixture in the mixing tank until the mud mixture achieves one or more predetermined rheological values. The method further includes supplying, using a density sensor, a weighting agent to the mud mixture in the mixing tank until the mud mixture achieves a predetermined specific gravity value to produce a drilling fluid.

In general, in one aspect, embodiments relate to a system that includes a mixing tank, various sensors coupled to the mixing tank, where the sensors include a rheological sensor and a density sensor. The system further includes a first control system coupled to the mixing tank and the sensors. The system further includes a drilling system coupled to the mixing tank and a wellbore, where the drilling system includes a second control system and a drill string. The second control system circulates drilling fluid within the wellbore. The first control system supplies diesel or mineral oil for a mud mixture to a mixing tank according to a predetermined volume. The first control system supplies an inorganic viscosifier, an emulsifier, a wetting agent, lime, a rheology modifier, a brine, and a fluid loss control additive to the mud mixture in the mixing tank. The first control system supplies, using a rheological sensor, a polymeric viscosifier to the mud mixture in the mixing tank until the mud mixture achieves one or more predetermined rheological values. The first control system supplies, using a density sensor, a weighting agent to the mud mixture in the mixing tank until the mud mixture achieves a predetermined specific gravity value to produce a drilling fluid.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIGS. 1 and 2 show systems in accordance with one or more embodiments.

FIGS. 3, 4, and 5 show flowcharts in accordance with one or more embodiments.

FIG. 6 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include systems and methods for automating a drilling fluid mixing process using real-time sensors and control systems. For example, various drilling fluid property values may be selected by a user or automatically by a particular control system. To achieve these drilling fluid property values, sensor data is collected at different stages of a mud mixing process. Once a specific value is achieved at one stage, the mud mixture may pass to the next stage for analysis and receiving additional additives. Thus, this automated mixing process may adjust a total mud volume and drilling fluid properties by adding fresh mud and additives throughout a continuous drilling operation. In some embodiments, for example, monitored drilling fluids properties may include rheological behavior, pH levels, and/or density values, such as specific gravity values, etc. Once a mud mixture achieves its desired drilling fluid property values, the mud mixture may be circulated through a drill string into the wellbore. Thus, automating a drilling fluid mixing process may eliminate health, safety, and environmental (HSE) issues, increase efficiency in drilling fluid production, increase drilling fluid quality, and/or reduce losses of net productive time (NPT).

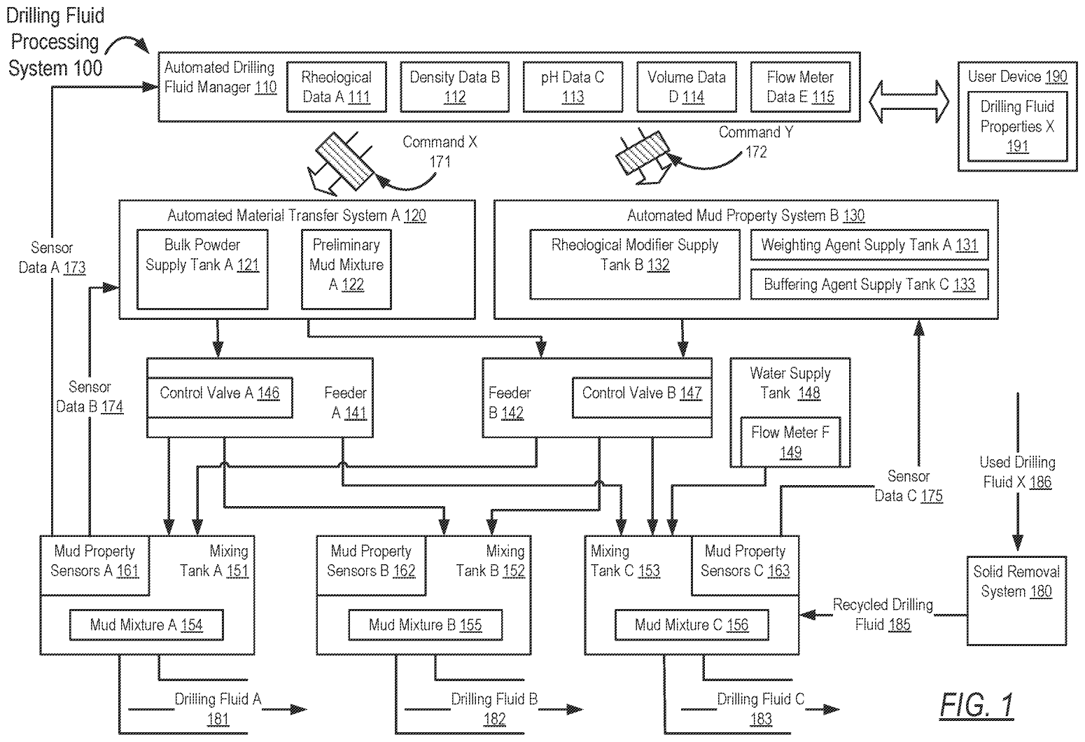

Turning to FIG. 1, FIG. 1 shows a schematic diagram in accordance with one or more embodiments. As shown in FIG. 1, FIG. 1 illustrates a drilling fluid processing system (100) that may include an automated drilling fluid manager (e.g., automated drilling fluid manager (110)) coupled to one or more user devices (e.g., user device (190)), various feeders (e.g., feeder A (141), feeder B (142)), various control valves (e.g., control valve A (146), control valve B (147)), various mixing tanks (e.g., mixing tank A (151), mixing tank B (152), mixing tank C (153)), and a solid removal system (e.g., solid removal system (180)). An automated drilling fluid manager may include hardware and/or software that includes functionality for monitoring and/or controlling various chemical components used within the drilling fluid processing system (100). In some embodiments, for example, the automated drilling fluid manager transmits one or more commands (e.g., command X (171), command Y (172)) to various control systems (e.g., automated material transfer system A (120), automated mud property system B (130)) in order to produce drilling fluids (e.g., drilling fluid A (181), drilling fluid B (182), drilling fluid C (183)) having specific drilling fluid properties (e.g., drilling fluid properties X (191)). Commands may include data messages transmitted over one or more network protocols using a network interface, such as through wireless data packets. Likewise, a command may also be a control signal, such as an analog electrical signal, that triggers one or more operations in a particular control system (e.g., automated mud property system B (130)) and/or a drilling fluid processing component (e.g., control valve A (146)).

Furthermore, drilling fluid properties may correspond to different physical qualities associated with drilling mud, such as specific gravity values, viscosity levels, pH levels, rheological values such as flow rates, temperature values, resistivity values, mud mixture weights, mud particle sizes, and various other attributes that affect the role of drilling fluid in a wellbore. For example, a drilling fluid property may be selected by a user device to have a desired predetermined value, which may include a range of acceptable values, a specific threshold value that should be exceeded, a precise scalar quantity, etc. As such, an automated drilling fluid manager or another control system may obtain sensor data (e.g., sensor data A (173), sensor data B (174), sensor data C (175)) from various mud property sensors (e.g., mud property sensors A (161), mud property sensors B (162), mud property sensors C (163)) regarding various drilling property parameters. Examples of mud property sensors include pH sensors, density sensors, rheological sensors, volume sensors, weight sensors, flow meters, such as an ES flow sensor, etc. Likewise, sensor data may refer to both raw sensor measurements and/or processed sensor data associated with one or more drilling fluid properties (e.g., rheological data A (111), density data B (112), pH data C (113), volume data D (114), flow meter data E (115)).

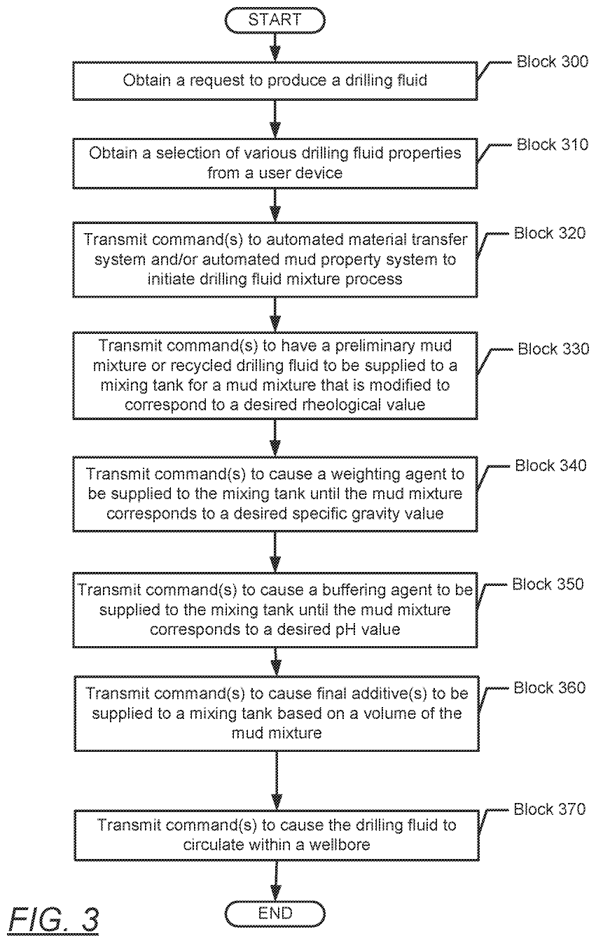

In some embodiments, an automated drilling fluid manager includes functionality for managing one or more mixing stages of a drilling fluid (e.g., mud mixture A (154), mud mixture B (155), mud mixture C (156)) within a mixing tank (e.g., mixing tank A (151), mixing tank B (152), mixing tank C (153)). A mixing stage may correspond to a single block from the workflows describes in FIGS. 3, 4, and 5 below, for example, as well as multiple blocks from a particular workflow. Furthermore, within this disclosure, “mud mixture” may refer to a mixture input or a mixture output of one or more mixing stages of a drilling fluid production process, while “drilling fluid” may refer to a final output of the drilling fluid production process that is sent to a wellbore. However, “mud mixture” and “drilling fluid” are not intended to have discrete meanings, and may be interchangeable at times, e.g., a “drilling fluid processing system” may manage “mud mixtures” at different stages of a “drilling fluid production process.”

With respect to a mixing tank, a mixing tank may be a container or other type of receptacle (e.g., a mud pit) for mixing various liquids, fresh mud, recycled mud, additives, and/or other chemicals to produce a particular mud mixture. For example, a mixing tank may be coupled to one or more mud supply tanks, one or more additive supply tanks, one or more dry/wet feeders (e.g., feeder A (141), feeder B (142)), and one or more control valves for managing the mixing of chemicals within a respective mixing tank. Control valves may be used to meter chemical inputs into a mixing tank, as well as release drilling fluid into a mixing tank. Likewise, a mixing tank may include and/or be coupled to various types of drilling fluid equipment not shown in FIG. 1, such as various mud pumps, mud lines, liquid supply lines, and/or other mixing equipment.

In some embodiments, the drilling fluid processing system (100) includes an automated material transfer system (e.g., automated material transfer system A (120)). In particular, an automated material transfer system may be a control system with functionality for managing supplies of bulk powder and other inputs for producing a preliminary mud mixture (e.g., preliminary mud mixture A (122)). For example, an automated material transfer system may include a pneumatic, conveyer belt or a screw-type transfer system (e.g., using a screw pump) that transports material from a supply tank upon a command from a sensor-mediated response. Once an automated drilling fluid manager actives an automated material transfer system, the automated material transfer system may monitor a mixing tank using weight sensors and/or volume sensors to meter a predetermined amount of bulk powder to a selected mixing tank. Likewise, the automated material transfer system may include hardware and/or software to provide water (e.g., using water supply tank (148)) to a mixing tank to produce a preliminary mud mixture. For example, water (or mineral oil or diesel in the case of oil-based muds) may be metered through a flow meter (e.g., flow meter F (149)) to a mixing tank. Once material for the preliminary mud mixture is transferred, the automated material transfer system may notify an automated drilling fluid manager that the task or mixing stage is complete. In some embodiments, for example, the preliminary mud mixture is a dry blended composition that has a predetermined accuracy with drilling fluid provided to a wellbore (e.g., drilling fluid properties that match 90-95% of a final mud mixture). For example, a preliminary mud mixture may be produced away from a wellsite and kept in a supply tank (e.g., bulk powder supply tank (121)) at the wellsite. Accordingly, an automated material transfer system may transfer additional amounts of materials from a supply tank upon command from a sensor-mediated response to fine-tune various drilling fluid properties.

Keeping with FIG. 1, once a preliminary mud mixture is disposed in a mixing tank, an automated drilling fluid manager may transmit one or more commands to activate an automated mud property system (e.g., automated mud property system B (130)) to control the supply of various additives to a mixing tank. In some embodiments, for example, an automated mud property system may include hardware and/or software with functionality for automatically supplying and/or mixing weighting agents (e.g., weighting agent supply tank A (131)), buffering agents (e.g., buffering agent supply tank C (133)), rheological modifiers (e.g., rheological modifier supply tank B (132)), and/or other additives until a mud mixture matches and/or satisfies one or more desired drilling fluid properties. Examples of weighting agents may include barite, hematite, calcium carbonate, siderite, etc. A buffering agent may be a pH buffering agent that causes a mud mixture to resist changes in pH levels. For example, a buffering agent may include water, a weak acid (or weak base) and salt of the weak acid (or a salt of weak base). Rheological modifiers may include drilling fluid additives that adjust one or more flow properties of a drilling fluid. One type of rheological modifier is a viscosifier, which may be an additive with functionality for providing thermal stability, hole-cleaning, shear-thinning, improving carrying capacity as well as modifying other attributes of a drilling fluid. Examples of viscosifiers include bentonite, inorganic viscosifiers, polymeric viscosifiers, low-temperature viscosifiers, high-temperature viscosifiers, oil-fluid liquid viscosifiers, organophilic clay viscosifiers, and biopolymer viscosifiers.

Furthermore, the automated mud property system and/or the automated drilling fluid manager may monitor various drilling fluid properties in real-time using one or more mud property sensors. In some embodiments, for example, the automated drilling fluid manager modifies drilling fluid properties of a mud mixture at predetermined intervals until user-defined drilling fluid properties are achieved by the drilling fluid processing system (100). The user-defined drilling fluid properties may correspond to a selection by a user device (e.g., drilling fluid properties X (191) obtained by user device (190)). For example, an automated drilling fluid manager may be coupled to a user device, e.g., through a user interface provided by the automated drilling fluid manager or remotely over a network (e.g., a remote connection through Internet access or a wireless connection at a well site). Based on real-time updates received for a current mud mixture, a user and/or the automated drilling fluid manager may also modify previously-selected drilling fluid property values during a mud mixing process, e.g., in response to changes in drilling operations in the wellbore.

Accordingly, once a mud mixture matches the drilling fluid properties and/or a mud mixture has completed one or more mixing stages, the automated mud property system may transmit a message acknowledging the current state of the mud mixture, e.g., the mud mixture is ready for a wellbore. Thus, the automated drilling fluid manager may include functionality for transmitting a command for causing drilling fluid to circulate through a drill string for continuous drilling, e.g., drilling fluid A (181), drilling fluid B (182), and drilling fluid C (183) shown in FIG. 1. Likewise, the drilling fluid processing system (100) may receive used drilling fluid from a wellbore (e.g. used drilling fluid X (186)) that is passed through a solid removal system (e.g., solid removal system (180)). More specifically, a solid removal system may include equipment and other hardware for removing particular solids, such as drill cuttings and coarse aggregates, from used drilling fluid in order to recycle drilling fluid (e.g., recycled drilling fluid (185)) into the drilling fluid processing system (100). Recycled drilling fluid may require fewer weighting agents, such as Barite, or mud additives for reprocessing prior to recirculation in a wellbore. Thus, an automated drilling fluid manager may also prepare recycled drilling fluid using an automated material transfer system and/or automated mud property system in a similar manner as performed for a fresh mud mixture.

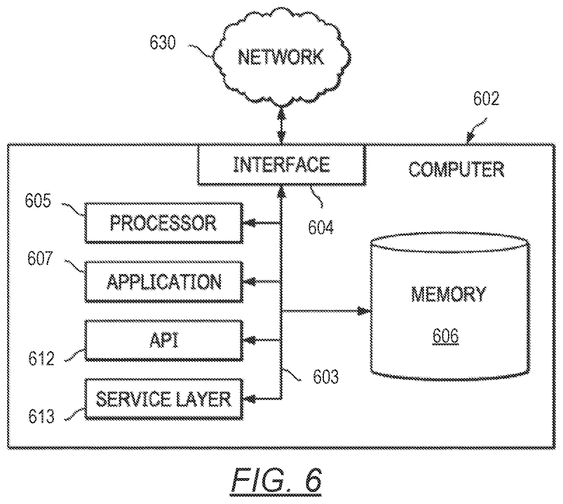

Keeping with FIG. 1, an automated drilling fluid manager, an automated material transfer system, and/or an automated mud property system may include one or more control systems that include one or more programmable logic controllers (PLCs). Specifically, a programmable logic controller may control valve states, fluid levels, pipe pressures, warning alarms, and/or pressure releases throughout a drilling fluid processing system (100). In particular, a programmable logic controller may be a ruggedized computer system with functionality to withstand vibrations, extreme temperatures, wet conditions, and/or dusty conditions, for example, around a drilling rig. In some embodiments, the automated drilling fluid manager (110), the automated material transfer system A (120), the automated mud property system B (130), and/or the user device (190) may include a computer system that is similar to the computer system (602) described below with regard to FIG. 6 and the accompanying description.

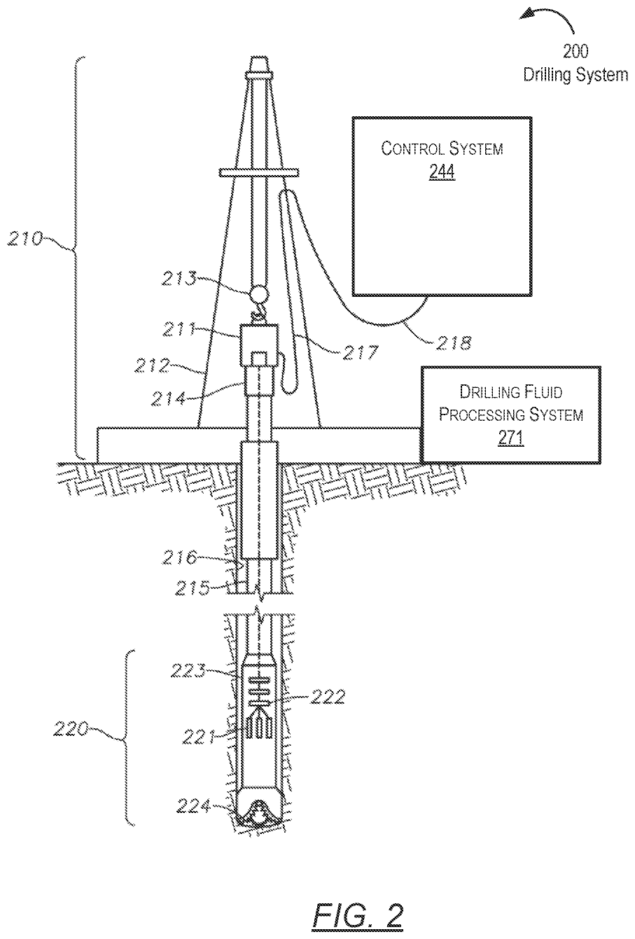

Turning to FIG. 2, FIG. 2 illustrate systems in accordance with one or more embodiments. As shown in FIG. 2, a drilling system (200) may include a top drive drill rig (210) arranged around the setup of a drill bit logging tool (220). A top drive drill rig (210) may include a top drive (211) that may be suspended in a derrick (212) by a travelling block (213). In the center of the top drive (211), a drive shaft (214) may be coupled to a top pipe of a drill string (215), for example, by threads. The top drive (211) may rotate the drive shaft (214), so that the drill string (215) and a drill bit logging tool (220) cut the rock at the bottom of a wellbore (216). A power cable (217) supplying electric power to the top drive (211) may be protected inside one or more service loops (218) coupled to a control system (244). As such, drilling fluid may be pumped into the wellbore (216) using a drilling fluid processing system (271), the drive shaft (214), and/or the drill string (215). The drilling fluid processing system (271) may be similar to the drilling fluid processing system (100) described above in FIG. 1 and the accompanying description. Likewise, the drilling fluid processing system may also include a mud pump, a mud line, mud pits, a mud return, and other components related to the circulation or recirculation of drilling fluid within the wellbore (216). The control system (244) may be similar to various control systems described above in FIG. 1 and the accompanying description, such as the automated material transfer system A (120) and/or the automated mud weight system B (130).

Moreover, when completing a well, casing may be inserted into the wellbore (216). The sides of the wellbore (216) may require support, and thus the casing may be used for supporting the sides of the wellbore (216). As such, a space between the casing and the untreated sides of the wellbore (216) may be cemented to hold the casing in place. The cement may be forced through a lower end of the casing and into an annulus between the casing and a wall of the wellbore (216). More specifically, a cementing plug may be used for pushing the cement from the casing. For example, the cementing plug may be a rubber plug used to separate cement slurry from other fluids, reducing contamination and maintaining predictable slurry performance. A displacement fluid, such as water, or an appropriately weighted drilling fluid, may be pumped into the casing above the cementing plug. This displacement fluid may be pressurized fluid that serves to urge the cementing plug downward through the casing to extrude the cement from the casing outlet and back up into the annulus.

As further shown in FIG. 2, sensors (221) may be included in a sensor assembly (223), which is positioned adjacent to a drill bit (224) and coupled to the drill string (215). Sensors (221) may also be coupled to a processor assembly (223) that includes a processor, memory, and an analog-to-digital converter (222) for processing sensor measurements. For example, the sensors (221) may include acoustic sensors, such as accelerometers, measurement microphones, contact microphones, and hydrophones. Likewise, the sensors (221) may include other types of sensors, such as transmitters and receivers to measure resistivity, gamma ray detectors, etc. The sensors (221) may include hardware and/or software for generating different types of well logs (such as acoustic logs or density logs) that may provide well data about a wellbore, including porosity of wellbore sections, gas saturation, bed boundaries in a geologic formation, fractures in the wellbore or completion cement, and many other pieces of information about a formation. If such well data is acquired during drilling operations (i.e., logging-while-drilling), then the information may be used to make adjustments to drilling operations in real-time. Such adjustments may include rate of penetration (ROP), drilling direction, altering mud weight, and many others drilling parameters.

In some embodiments, acoustic sensors may be installed in a drilling fluid circulation system of a drilling system (200) to record acoustic drilling signals in real-time. Drilling acoustic signals may transmit through the drilling fluid to be recorded by the acoustic sensors located in the drilling fluid circulation system. The recorded drilling acoustic signals may be processed and analyzed to determine well data, such as lithological and petrophysical properties of the rock formation. This well data may be used in various applications, such as steering a drill bit using geosteering, casing shoe positioning, etc.

The control system (244) may be coupled to the sensor assembly (223) in order to perform various program functions for up-down steering and left-right steering of the drill bit (224) through the wellbore (216). More specifically, the control system (244) may include hardware and/or software with functionality for geosteering a drill bit through a formation in a lateral well using sensor signals, such as drilling acoustic signals or resistivity measurements. For example, the formation may be a reservoir region, such as a pay zone, bed rock, or cap rock.

Turning to geosteering, geosteering may be used to position the drill bit (224) or drill string (215) relative to a boundary between different subsurface layers (e.g., overlying, underlying, and lateral layers of a pay zone) during drilling operations. In particular, measuring rock properties during drilling may provide the drilling system (200) with the ability to steer the drill bit (224) in the direction of desired hydrocarbon concentrations. As such, a geo steering system may use various sensors located inside or adjacent to the drilling string (215) to determine different rock formations within a wellbore' s path. In some geosteering systems, drilling tools may use resistivity or acoustic measurements to guide the drill bit (224) during horizontal or lateral drilling.

While FIGS. 1 and 2 shows various configurations of components, other configurations may be used without departing from the scope of the disclosure. For example, various components in FIGS. 1 and 2 may be combined to create a single component. As another example, the functionality performed by a single component may be performed by two or more components.

Turning to FIG. 3, FIG. 3 shows a flowchart in accordance with one or more embodiments. Specifically, FIG. 3 describes a general method for producing drilling fluid. One or more blocks in FIG. 3 may be performed by one or more components (e.g., automated drilling fluid manager (110)) as described in FIGS. 1 and 2. While the various blocks in FIG. 3 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 300, a request is obtained to produce a drilling fluid in accordance with one or more embodiments. In some embodiments, a user device transmits a request to a drilling fluid processing system to initiate an automated sequence for preparing drilling fluid. For example, a user may select an icon from a graphical user interface that triggers an automated drilling fluid manager to begin transmitting commands to one or more components within the drilling fluid processing system. Likewise, in some embodiments, an automated drilling fluid manager initiates the automated sequence in response to one or more events detected at a drilling rig, such as detected changes in a drilling operation.

In Block 310, a selection of various drilling fluid properties is obtained from a user device in accordance with one or more embodiments. For example, a user may select values for different drilling fluid properties. This selection may be part of the request transmitted in Block 300 above. In another example, a user device or a control system in a drilling rig may determine a specific type of drilling operation that requires a specific type of drilling fluid, e.g., based on a formation type or a particular well path design. Accordingly, the drilling fluid properties may be automatically selected in response to determining the particular type of drilling operation.

In Block 320, one or more commands are transmitted to an automated material transfer system and/or an automated mud property system in accordance with one or more embodiments. Once an automated sequence for producing drilling fluid is initiated, an automated drilling fluid manager may transmit commands to one or more control systems, such as an automated material transfer system and/or automated mud property system. These commands may identify values of different drilling fluid properties as well as a selection of different mixing stages for a drilling fluid. For example, these commands may identify whether a sequence for a water-based mud or an oil-based mud is used for producing the drilling fluid. Thus, different control systems may be notified of specific drilling fluid properties before mixing begins on any chemical compounds of the drilling fluid. On the other hand, an automated drilling fluid manager may also transmit commands throughout a mixing process to update different control systems on changes to a mud mixture, e.g., in response to real-time sensor data or drilling operation events.

In Block 330, one or more commands are transmitted that cause a preliminary mud mixture or recycled drilling fluid to be supplied to a mixing tank for a mud mixture that is modified to correspond to a desired rheological value in accordance with one or more embodiments. For example, an automated material transfer system and/or an automated mud property system may operate to produce a preliminary mud mixture with one or more rheological values corresponding to at least one selected value. With respect to recycled drilling fluid, recycled drilling fluid may drilling fluid properties within a predetermined accuracy with final drilling fluid (e.g., equivalent to 90% good mud). Thus, an automated drilling fluid manager may fine-tune recycled drilling fluid by modifying the resulting mud mixture to achieve desired drilling fluid properties. This modification may be performed in Blocks 330, 340, 350, and/or 360, for example, by adding any needed materials based on sensor responses.

In some embodiments, a rheological sensor is used to monitor a mud mixture based on the preliminary mud mixture or recycled drilling fluid to achieve a desired rheological value of a mud mixture. For example, the rheological value may correspond to a predetermine yield point to plastic viscosity ratio. Sensor data may be periodically obtained from a mixing tank to determine whether the rheological value has been satisfied. Where the rheological value has yet to be achieved, a rheological modifier may be continually added to the mixing tank.

In some embodiments, for example, the preliminary mud mixture is a single viscosifier, such as bentonite, or an inorganic viscosifier for an oil-based mud. In other embodiments, the preliminary mud mixture is a dry blend mud (DBM) or a minimum additive mud (MAM) for a water-based mud. With respect to a dry blend mud, a dry blend mixture may be a physical blend of various mud components such as a low temp viscosifier, a high temp viscosifier, a rheology modifier, a filtrate loss control additive and a thinner in appropriate ratios and capable of providing a preliminary mud mixture for a mixing tank. In the case of a water-based mud (WBM), the preliminary mud mixture may be at a density range from 8 parts per gallon (ppg) to 22 ppg after mixing with water and Barite in particular amounts. In some embodiments, a drilling fluid parameter corresponds to a predetermined ratio between the preliminary mud mixture, water, and Barite to produce a mud mixture at a predetermined density. With respect to a minimum additive mud, a MAM mixture may be capable of providing a preliminary mud mixture of a water-based mud at a range of densities from 8 ppg to 22 ppg after mixing the water and barite in predetermined amounts. A MAM mixture may be processed outside of a wellsite in a similar manner as other preliminary mud mixtures. In some embodiments, a MAM mixture includes a predetermined minimum number of additives (e.g., no more than four additives) to produce the final drilling fluid. Therefore, a MAM mixture may increase the production rate of an automated mixing process.

In Block 340, one or more commands are transmitted that cause a weighting agent to be supplied to a mixing tank until the mud mixture corresponds to a desired specific gravity value in accordance with one or more embodiments. In some embodiments, for example, a weighting agent, such as barite, may be continually added to a mud mixture until a predetermined density or specific gravity is achieved. Here, a density sensor may be coupled to a mixing tank to obtain density data of the mud mixture. Similar to Block 330, an automated mud property system and/or automated drilling fluid manager may automatically monitor the mud mixture until a predetermined specific gravity value or other density-related value is achieved based on a selected value.

In Block 350, one or more commands are transmitted that cause a buffering agent to be supplied to a mixing tank until a mud mixture corresponds to a desired PH value in accordance with one or more embodiments. Similar to Blocks 330 and 340 above, a buffering agent may be added to a mixing tank until the pH value of the mud mixture satisfies a selected drilling fluid parameter.

In Block 360, one or more commands are transmitted that cause one or more final additives to be supplied to a mixing tank based on a volume of a mud mixture in accordance with one or more embodiments. Once a mud mixture has achieved desired rheological properties, density properties, and pH properties, one or more final additives may be added to the mud mixture in order to complete the mixing process for producing drilling fluid. For example, the current volume of a mud mixture may be determined using volume sensors or based on flow measurements of previous chemical inputs applied to the mixing tank.

In Block 370, one or more commands are transmitted to cause a drilling fluid to circulate within a wellbore in accordance with one or more embodiments.

### Automated Process for Water-Based Mud (WBM)

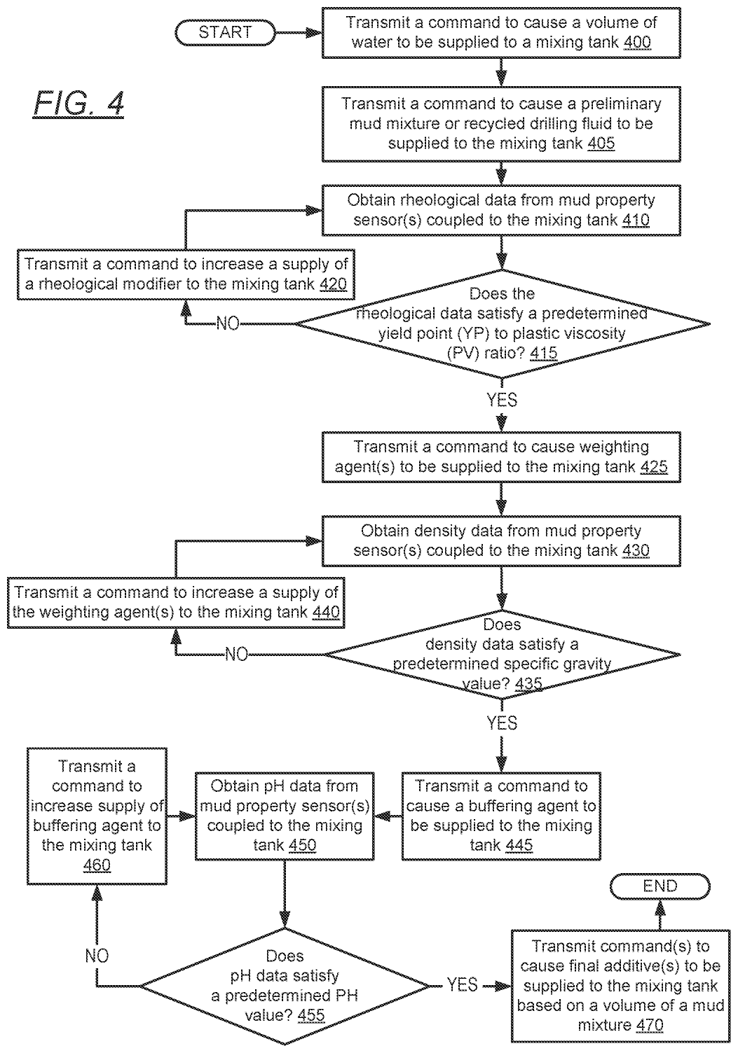

Turning to FIG. 4, FIG. 4 shows a flowchart in accordance with one or more embodiments. Specifically, FIG. 4 describes a specific method for producing a drilling fluid from a water-based mud mixture. One or more blocks in FIG. 4 may be performed by one or more components (e.g., automated drilling fluid manager (110)) as described in FIGS. 1 and 2. While the various blocks in FIG. 4 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 400, a command is transmitted that causes a volume of water to be supplied to a mixing tank in accordance with one or more embodiments. For the drilling fluid procedure, a mixing tank may begin with a predetermined volume of water. For example, the volume of water may be determined based on the volume of drilling fluid desired for a particular wellbore or drilling operation.

In Block 405, a command is transmitted that causes a preliminary mud mixture or recycled drilling fluid to be supplied to a mixing tank in accordance with one or more embodiments. For example, the preliminary mud mixture may be a single viscosifier, such as bentonite, a dry blend mud, or a minimum additive mud as described above in Block 330 and the accompanying description.

In Block 410, rheological data are obtained from one or more mud property sensors coupled to a mixing tank in accordance with one or more embodiments. The rheological data may be sensor data from a rheological sensor as described above in FIG. 1 and the accompanying description.

In Block 415, a determination is made whether rheological data satisfy a predetermined yield point to plastic viscosity ratio in accordance with one or more embodiments. For example, an automated mud property system may analyze rheological data regarding a mud mixture to determine whether a YP/PV ratio or other rheological parameter corresponds to a selected value. Where a determination is made that the rheological data satisfies a predetermined rheological value, such as a YP/PV ratio, the process shown in FIG. 4 may proceed to Block 425. Where a determination is made that the mud mixture has not achieved the predetermined rheological value, the process shown in FIG. 4 may proceed to Block 420.

In Block 420, a command is transmitted to increase a supply of a rheological modifier to a mixing tank in accordance with one or more embodiments. For example, a preliminary mud mixture may be converted into an intermediate mud mixture by fine-tuning one or more rheological parameters upon addition of a controlled addition of liquid version of one or more rheology modifiers. In some embodiments, the rheology modifier is a diluted version in contrast to the viscosifier used to produce the preliminary mud mixture.

In Block 425, a command is transmitted that causes one or more weighting agents to be supplied to a mixing tank in accordance with one or more embodiments. Based on initial sensor data from a density sensor, a predetermined amount of weighting agent may be supplied to a mixing tank with a mud mixture.

In Block 430, density data are obtained from one or more mud property sensors coupled to a mixing tank in accordance with one or more embodiments.

In Block 435, a determination is made whether density data satisfy a predetermined specific gravity value in accordance with one or more embodiments. For example, an automated mud property system may analyze density data regarding a mud mixture to determine whether a specific gravity or other density-related parameter corresponds to a selected value for a drilling fluid. Where a determination is made that the density data satisfies a predetermined specific gravity value, the process shown in FIG. 4 may proceed to Block 445. Where a determination is made that the mud mixture has not achieved the predetermined rheological value, the process shown in FIG. 4 may proceed to Block 440.

In Block 440, a command is transmitted to increase a supply of one or more weighting agents to a mixing tank in accordance with one or more embodiments.

In Block 445, a command is transmitted that causes a buffering agent to be supplied to a mixing tank in accordance with one or more embodiments.

In Block 450, pH data are obtained from one or more mud property sensors coupled to the mixing tank in accordance with one or more embodiments.

In Block 455, a determination is made whether pH data satisfy a predetermined pH value in accordance with one or more embodiments. For example, an automated mud property system may analyze pH data regarding a mud mixture to determine whether a pH value corresponds to a selected value for a drilling fluid. Where a determination is made that the pH data satisfies a predetermined pH value, the process shown in FIG. 4 may proceed to Block 470. Where a determination is made that the mud mixture has not achieved the predetermined pH value, the process shown in FIG. 4 may proceed to Block 460.

In Block 460, a command is transmitted to increase a supply of a buffering agent to a mixing tank in accordance with one or more embodiments.

In Block 470, a command is transmitted to cause one or more final additives to be supplied to a mixing tank based on a volume of a mud mixture in accordance with one or more embodiments. For example, an automated mud property system may obtain volume measurements using a volume sensor to determine various quantities of oxygen scavenger, sour gas scavenger, shale inhibiters, lubricants, etc. to add to a mud mixture to complete a drilling fluid production process.

### Automated Process for Oil-Based Mud (OBM)

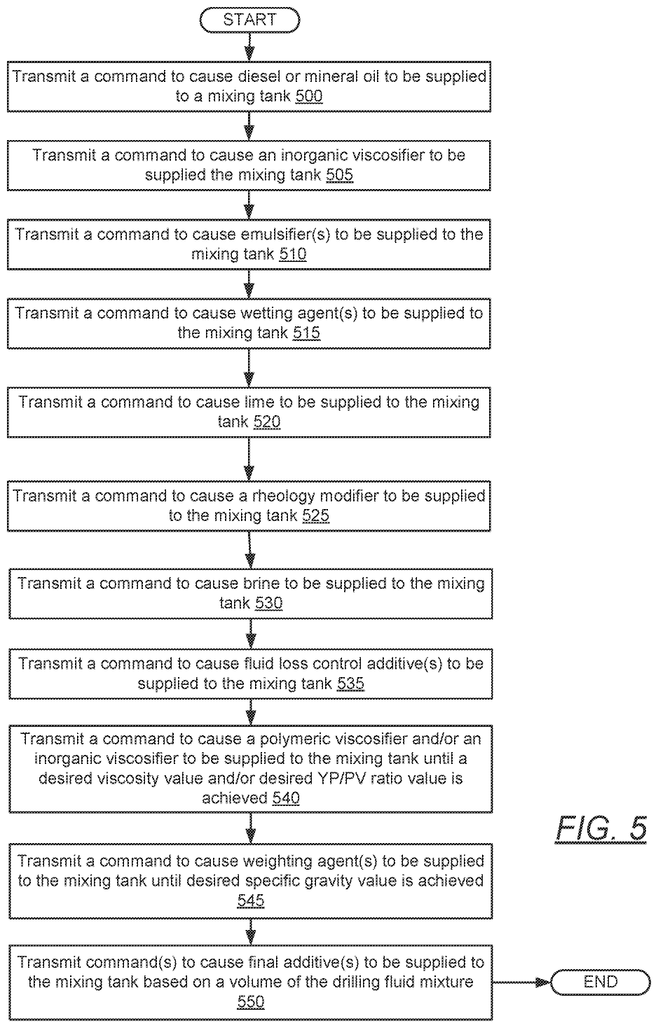

Turning to FIG. 5, FIG. 5 shows a flowchart in accordance with one or more embodiments. Specifically, FIG. 5 describes a specific method for producing a drilling fluid from an oil-based mud mixture. One or more blocks in FIG. 5 may be performed by one or more components (e.g., automated drilling fluid manager (110)) as described in FIGS. 1 and 2. While the various blocks in FIG. 5 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 500, a command is transmitted that causes diesel or mineral oil to be supplied to a mixing tank in accordance with one or more embodiments. In some embodiments, recycled drilling fluid may be used to produce a preliminary mud mixture in place of diesel or mineral oil. For example, the recycled drilling fluid may be supplied to a mixing tank after removing cutting/solids using a solid removal system.

In Block 505, a command is transmitted that causes an inorganic viscosifier to be supplied to a mixing tank in accordance with one or more embodiments.

In Block 510, a command is transmitted that causes one or more emulsifiers to be supplied to a mixing tank in accordance with one or more embodiments. An emulsifier may be a chemical used in producing an oil-based or synthetic oil-based drilling fluid that forms a water-in-oil emulsion. In particular, an emulsifier may lower the interfacial tension between oil and water. For example, emulsifiers may be a primary emulsifier or a secondary emulsifier, where the secondary emulsifier is rarely used alone in producing a drilling fluid. Emulsifiers may include calcium fatty-acid soaps made from various fatty acids and lime, and/or derivatives such as amides, amines, amidoamines and imidazolines made by reactions of fatty acids, and various ethanolamine compounds.

In Block 515, a command is transmitted that causes one or more wetting agents to be supplied to a mixing tank in accordance with one or more embodiments. A wetting agent may be a surfactant that reduces various sticking tendencies of clay and shale. For example, a wetting agent may reduce the formation of mud rings in a drilling fluid. Baroid is an example of a wetting agent.

In Block 520, a command is transmitted that causes lime to be supplied to a mixing tank in accordance with one or more embodiments.

In Block 525, a command is transmitted that causes a rheological modifier to be supplied to a mixing tank in accordance with one or more embodiments. A rheological modifier may be similar to the rheological modifiers describes above in FIG. 1 and in Block 330 in FIG. 3 and the accompanying description.

In Block 530, a command is transmitted that causes brine to be supplied to a mixing tank in accordance with one or more embodiments. A “brine” may refer to salts and salt mixtures dissolved in a mud mixture. More specifically, brine may be a solution of sodium chloride, such as an emulsified calcium chloride solution (or any other saline phase solution).

In Block 535, a command is transmitted that causes one or more fluid loss control additives to be supplied to a mixing tank in accordance with one or more embodiments. A fluid loss control additive may be a drilling fluid additive with functionality for lowering the volume of filtrate that passes through a filter medium.

In Block 540, a command is transmitted that causes a polymeric viscosifier and/or an inorganic viscosifier to be supplied to a mixing tank until a desired viscosity value and/or a desired YP/PV ratio value are achieved in accordance with one or more embodiments. For example, an automated mud property system may achieve a predetermined rheological value with a mud mixture by adding a polymeric viscosifier and/or an inorganic visocosifier in a similar manner as described above in Blocks 410, 415, and 420 in FIG. 4 and the accompanying description.

In Block 545, a command is transmitted that causes one or more weighting agents to be supplied to a mixing tank until a desired specific gravity value is achieved in accordance with one or more embodiments. For example, an automated mud property system may achieve a predetermined specific gravity value with a mud mixture by adding a weighting agent in a similar manner as described above in Blocks 425, 430, 435, and 440 in FIG. 4 and the accompanying description.

In Block 550, a command is transmitted that causes one or more final additives to be supplied to a mixing tank in accordance with one or more embodiments. For example, an automated mud property system may obtain volume measurements using a volume sensor to determine various quantities of oxygen scavenger, sour gas scavenger, shale inhibiters, lubricants, etc. to add to a mud mixture to complete a drilling fluid production process.

### Computer System

Embodiments may be implemented on a computer system. FIG. 6 is a block diagram of a computer system (602) used to provide computational functionalities associated with described algorithms, methods, functions, processes, flows, and procedures as described in the instant disclosure, according to an implementation. The illustrated computer (602) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (602) may include a computer that includes an input device, such as a keypad, keyboard, touch screen, or other device that can accept user information, and an output device that conveys information associated with the operation of the computer (602), including digital data, visual, or audio information (or a combination of information), or a GUI.

The computer (602) can serve in a role as a client, network component, a server, a database or other persistency, or any other component (or a combination of roles) of a computer system for performing the subject matter described in the instant disclosure. The illustrated computer (602) is communicably coupled with a network (630). In some implementations, one or more components of the computer (602) may be configured to operate within environments, including cloud-computing-based, local, global, or other environment (or a combination of environments).

At a high level, the computer (602) is an electronic computing device operable to receive, transmit, process, store, or manage data and information associated with the described subject matter. According to some implementations, the computer (602) may also include or be communicably coupled with an application server, e-mail server, web server, caching server, streaming data server, business intelligence (BI) server, or other server (or a combination of servers).

The computer (602) can receive requests over network (630) from a client application (for example, executing on another computer (602)) and responding to the received requests by processing the said requests in an appropriate software application. In addition, requests may also be sent to the computer (602) from internal users (for example, from a command console or by other appropriate access method), external or third-parties, other automated applications, as well as any other appropriate entities, individuals, systems, or computers.

Each of the components of the computer (602) can communicate using a system bus (603). In some implementations, any or all of the components of the computer (602), both hardware or software (or a combination of hardware and software), may interface with each other or the interface (604) (or a combination of both) over the system bus (603) using an application programming interface (API) (612) or a service layer (613) (or a combination of the API (612) and service layer (613). The API (612) may include specifications for routines, data structures, and object classes. The API (612) may be either computer-language independent or dependent and refer to a complete interface, a single function, or even a set of APIs. The service layer (613) provides software services to the computer (602) or other components (whether or not illustrated) that are communicably coupled to the computer (602). The functionality of the computer (602) may be accessible for all service consumers using this service layer. Software services, such as those provided by the service layer (613), provide reusable, defined business functionalities through a defined interface. For example, the interface may be software written in JAVA, C++, or other suitable language providing data in extensible markup language (XML) format or other suitable format. While illustrated as an integrated component of the computer (602), alternative implementations may illustrate the API (612) or the service layer (613) as stand-alone components in relation to other components of the computer (602) or other components (whether or not illustrated) that are communicably coupled to the computer (602). Moreover, any or all parts of the API (612) or the service layer (613) may be implemented as child or sub-modules of another software module, enterprise application, or hardware module without departing from the scope of this disclosure.

The computer (602) includes an interface (604). Although illustrated as a single interface (604) in FIG. 6, two or more interfaces (604) may be used according to particular needs, desires, or particular implementations of the computer (602). The interface (604) is used by the computer (602) for communicating with other systems in a distributed environment that are connected to the network (630). Generally, the interface (604 includes logic encoded in software or hardware (or a combination of software and hardware) and operable to communicate with the network (630). More specifically, the interface (604) may include software supporting one or more communication protocols associated with communications such that the network (630) or interface's hardware is operable to communicate physical signals within and outside of the illustrated computer (602).

The computer (602) includes at least one computer processor (605). Although illustrated as a single computer processor (605) in FIG. 6, two or more processors may be used according to particular needs, desires, or particular implementations of the computer (602). Generally, the computer processor (605) executes instructions and manipulates data to perform the operations of the computer (602) and any algorithms, methods, functions, processes, flows, and procedures as described in the instant disclosure.

The computer (602) also includes a memory (606) that holds data for the computer (602) or other components (or a combination of both) that can be connected to the network (630). For example, memory (606) can be a database storing data consistent with this disclosure. Although illustrated as a single memory (606) in FIG. 6, two or more memories may be used according to particular needs, desires, or particular implementations of the computer (602) and the described functionality. While memory (606) is illustrated as an integral component of the computer (602), in alternative implementations, memory (606) can be external to the computer (602).

The application (607) is an algorithmic software engine providing functionality according to particular needs, desires, or particular implementations of the computer (602), particularly with respect to functionality described in this disclosure. For example, application (607) can serve as one or more components, modules, applications, etc. Further, although illustrated as a single application (607), the application (607) may be implemented as multiple applications (607) on the computer (602). In addition, although illustrated as integral to the computer (602), in alternative implementations, the application (607) can be external to the computer (602).

There may be any number of computers (602) associated with, or external to, a computer system containing computer (602), each computer (602) communicating over network (630). Further, the term “client,” “user,” and other appropriate terminology may be used interchangeably as appropriate without departing from the scope of this disclosure. Moreover, this disclosure contemplates that many users may use one computer (602), or that one user may use multiple computers (602).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, any means-plus-function clauses are intended to cover the structures described herein as performing the recited function(s) and equivalents of those structures.

Similarly, any step-plus-function clauses in the claims are intended to cover the acts described here as performing the recited function(s) and equivalents of those acts. It is the express intention of the applicant not to invoke 35 U.S.C. § 112(f) for any limitations of any of the claims herein, except for those in which the claim expressly uses the words “means for” or “step for” together with an associated function.

What is claimed:

1. A method, comprising:
supplying water for a mud mixture to a mixing tank according to a predetermined volume;
supplying, using a rheological sensor, a viscosifier to the mud mixture in the mixing tank until the mud mixture achieves one or more predetermined rheological values;
supplying, using a density sensor, a weighting agent to the mud mixture in the mixing tank until the mud mixture achieves a predetermined specific gravity value; and
supplying, using a pH sensor, a buffering agent to the mud mixture in the mixing tank until the mud mixture achieves a predetermined pH value to produce a drilling fluid.

2. The method of claim 1, further comprising:
supplying the drilling fluid to a wellbore.

3. The method of claim 1, further comprising:
obtaining, by a user device, a selection of a plurality of drilling fluid properties,
wherein the plurality of drilling fluid properties comprise the one or more predetermined rheological values, the predetermined specific gravity value, and the predetermined pH value.

4. The method of claim 1,
wherein the one or more predetermined rheological values comprises a predetermined ratio between a yield point (YP) to a plastic viscosity (PV).

5. The method of claim 1,
wherein the predetermined volume is determined using a volume sensor coupled to the mixing tank, and
wherein the water, the first viscosifier, the weighting agent, and a buffering agent are supplied to the mixing tank in this order to produce a water-based mud (WBM) mixture.

6. The method of claim 1, further comprising:
supplying one or more final additives to the mud mixture based on a volume measurement of the mud mixture,
wherein the one or more final additives are selected from a group consisting of an oxygen scavenger, a sour gas scavenger, a lubricant, and a shale inhibiter.

7. The method of claim 1,
wherein the rheological sensor, the density sensor, and the pH sensor transmit sensor data to a control system in real-time.

8. The method of claim 1,
wherein the first viscosifier is part of a dry blend mixture comprising an rheology modifier, a second viscosifier, a filtrate loss control additive, and a thinner, and
wherein the dry blend mixture is mixed with the water and Barite in a predetermined ratio to produce a preliminary mud mixture for the mixing tank.

9. The method of claim 1,
wherein the first viscosifier is part of a minimum additive mud (MAM) mixture, and
wherein the MAM is mixed with the water and Barite in a predetermined ratio to produce a preliminary mud mixture for the mixing tank.

10. A system, comprising:
a mixing tank;
a plurality of sensors coupled to the mixing tank, the plurality of sensors comprising a rheological sensor, a density sensor, and a pH sensor;
a first control system coupled to the mixing tank and the plurality of sensors;
a drilling system coupled to the mixing tank and a wellbore, the drilling system comprising a second control system and a drill string, and
wherein the first control system comprises functionality for:
supplying water for a mud mixture to a mixing tank according to a predetermined volume;
supplying, using the rheological sensor, a viscosifier to the mud mixture in the mixing tank until the mud mixture achieves one or more predetermined rheological values;
supplying, using the density sensor, a weighting agent to the mud mixture in the mixing tank until the mud mixture achieves a predetermined specific gravity value; and
supplying, using the pH sensor, a buffering agent to the mud mixture in the mixing tank until the mud mixture achieves a predetermined pH value to produce a drilling fluid.

11. The system of claim 10, further comprising:
a user device coupled to the first control system,
wherein the first control system comprises functionality for obtaining, from the user device, a selection of a plurality of drilling fluid properties,
wherein the plurality of drilling fluid properties comprise the one or more predetermined rheological values, the predetermined specific gravity value, and the predetermined pH value.

12. The system of claim 10, further comprising:
a supply tank coupled to the mixing tank,
wherein the supply tank stores dry blend mixture for producing a preliminary mud mixture in the mixing tank, and
wherein the dry blend mixture comprises a rheology modifier, a second viscosifier, a filtrate loss control additive, and a thinner.

13. The system of claim 10, further comprising:
a user device coupled to the first control system,
wherein the first control system comprises functionality for obtaining, from the user device, a selection of a plurality of drilling fluid properties,
wherein the plurality of drilling fluid properties comprise the one or more predetermined rheological values, the predetermined specific gravity value, and the predetermined pH value.

14. The system of claim 10,
wherein the rheological sensor, the density sensor, and the pH sensor transmit sensor data to the first control system in real-time.

15. A method, comprising:
supplying diesel or mineral oil to a mixing tank according to a predetermined volume to produce a mud mixture;
supplying an inorganic viscosifier, one or more emulsifiers, a wetting agent, lime, a rheology modifier, a brine, and a fluid loss control additive to the mud mixture in the mixing tank;
supplying, using a rheological sensor, a polymeric viscosifier to the mud mixture in the mixing tank until the mud mixture achieves one or more predetermined rheological values; and
supplying, using a density sensor, a weighting agent to the mud mixture in the mixing tank until the mud mixture achieves a predetermined specific gravity value to produce a drilling fluid.

16. The method of claim 15, further comprising:
supplying the drilling fluid to a wellbore.

17. The method of claim 15, further comprising:
obtaining, by a user device, a selection of a plurality of drilling fluid properties,
wherein the plurality of drilling fluid properties comprise the one or more predetermined rheological values and the predetermined specific gravity value.

18. The method of claim 15, further comprising:
supplying one or more final additives to the mud mixture based on a volume measurement of the mud mixture,
wherein the one or more final additives are selected from a group consisting of an oxygen scavenger, a sour gas scavenger, a lubricant, and a shale inhibiter.

19. The method of claim 15,
wherein the rheological sensor, the density sensor, and the pH sensor transmit sensor data to a control system in real-time.

20. The method of claim 15,
wherein the diesel or the mineral oil comprises recycled drilling fluid that is obtained from a wellbore.
